(19) United States
(12) Patent Application Publication
(10) Pub. No.: US 2010/0331854 A1
(43) Pub. Date: Dec. 30, 2010

(54) DEVICE AND METHOD FOR PERFORMING TREATMENT IN A PERICARDIAL SPACE

(76) Inventors: Ilan Greenberg, Haifa (IL); Gideon Meiry, Kibbutz Shomrat (IL)

Correspondence Address: Shiloh et al., 99 John St., Suite 2303, New York, NY, 10038, US

(21) Appl. No.: 12/865,899

(22) PCT Filed: Jan. 7, 2009
(86) PCT No.: PCT/IB09/50029

§ 371 Date: Aug. 3, 2010

Related U.S. Application Data
(60) Provisional application No. 61/006,323, filed on Jan. 7, 2008.

(51) Int. Cl. A61M 25/09; A61M 25/04; A61M 25/06; A61B 19/00

(52) U.S. Cl. 606/129; 604/175

(57) ABSTRACT

An apparatus, device and method are provided to enable performing a treatment in a minimally invasive fashion in a pericardial space. The device includes a needle having a sensing means to detect substantially different bodily tissues to enable a practitioner to determine when the distal end of the needle is in the pericardial space; an anchoring means to secure the distal end of the needle inside or in proximity to the pericardial space; and a delivery means for delivering a guidewire to the pericardial space to perform the treatment.

### CROSS REFERENCE TO RELATED APPLICATIONS

This application claims priority from U.S. Provisional Patent Application No. 61/006,323, filed Jan. 07, 2008, entitled “DEVICE FOR LOCATING AND ACCESSING A PERICARDIAL SPACE”, which is incorporated in its entirety herein by reference.

### FIELD OF THE INVENTION

This invention relates to minimally invasive cardiac procedures. More specifically, the invention relates to a device and method for locating and performing treatment in or around the pericardial cavity surrounding the heart.

### BACKGROUND OF THE INVENTION

Congestive Heart Failure (CHF) is a common clinical syndrome resulting from heart damage, wherein the metabolic demands of the body are not met by the output of the heart. Approximately 15% of heart failure patients have an inter-and/or-intra Ventricular Conduction Delay (IVCD) typically resulting from left bundle branch block (LBBB). LBBB typically results in asynchronous activation and contraction of the ventricles. Ventricular desynchrony has several important consequences that adversely affect cardiac performance. Among these are Abnormal inter-ventricular septal wall motion, Reduced diastolic filling time, Mitral regurgitation, and Reduced dP/dt. CHF patients with ventricular desynchrony suffer from continuous deterioration in their cardiac performance, reduction in their quality of life and increased mortality rates.

A recent approach aimed at treating cardiac desynchrony is known as “cardiac resynchronization therapy” (CRT), or biventricular pacing. CRT uses a specialized pacemaker to re-coordinate the contraction of the right and left ventricles. When used in combination with stable, optimal medical therapy, CRT is designed to reduce symptoms by restoring cardiac synchronicity. CRT typically provides atrial-synchronized, biventricular pacing using right atria and right ventricle standard pacing leads technology combined with a special third lead which is implanted via the coronary sinus and positioned in a cardiac vein to sense and pace the left ventricle. Following a sensed atrial activation, both ventricles are stimulated to contract simultaneously. ECG and echocardiography data from clinical trials have shown improved cardiac function as a result of CRT. Improved activation sequence and contraction timing appear to be the two primary mechanisms by which CRT enhances cardiac performance in patients with IVCD.

The success of CRT in patients with cardiac desynchrony depends substantially on left ventricle (LV) electrode positioning. Imaging tools such as tissue Doppler imaging (TDI) enables the physician to identify late contracting areas in the LV. Pacing such late contracting areas preempts their electrical and therefore mechanical activity, and by doing so, resynchronizes ventricular activity and improves overall cardiac performance. Successful CRT requires the ability to: Determine optimal pacing site(s); Access optimal pacing site(s); Position left-heart lead(s) at the optimal pacing site(s); and Stabilize the left-heart lead(s) in the optimal site(s).

However, the current methodology of CRT suffers from several problems, in particular problems associated with pacing the left ventricle, including reaching optimal pacing sites in the left ventricle, which may be dependent on and therefore restricted by the anatomy of the cardiac venous system; and catheterization of the coronary sinus and positioning the LV electrode, which is often a long procedure lasting several hours, therefore posing various safety risks. Furthermore, in general, CRT is needed in elderly patients in which coronary sinus catheterization is sometime problematic. Moreover, the current approach is limited to placing only one left ventricular electrode, which is generally not efficient for treating left ventricle intra conduction delays.

Due to these and other limitations, current CRT fails in approximately 40% of the patients. Discordance between the site(s) of maximal delay and the sites of pacing is accepted to be the main reason for the lack of benefit in so many patients.

WO2007/017879, filed by the current inventor, discloses an approach and tools for CRT pacing in which the left side of the heart is paced by epicardial electrode(s) which replace the endocardial transvenous left side lead. The epicardial leads are introduced into the pericardial cavity by a percutaneous minimally invasive procedure, and are navigated and placed at specific epicardial locations to give a more accurate synchronizing pacing treatment. The major challenge in procedures requiring minimally invasive access into the pericardial cavity is accurately locating the pericardial sac and/or cavity and administering treatment within or adjacent to the pericardial cavity. The pericardium is a double-walled sac that contains the heart and the roots of the great vessels. The sac is composed of two layers: the outer fibrous pericardium and the inner serous pericardium. The fibrous pericardium contains dense connective tissue that protects the heart, anchors it to the surrounding walls, and prevents it from overfilling with blood. The serous pericardium is divided into two layers, the parietal pericardium, which is fused to, and inseparable from, the fibrous pericardium which together compose the pericardial sac, and the visceral pericardium, which is fused to the outer surface of the heart muscle, the epicardium. In between the parietal and visceral pericardial layers lies a potential space called the pericardial cavity. Normally, the cavity contains pericardial fluid that provides lubrication to the heart movements inside the pericardial sac.

The pericardial cavity is a target for various medical treatments. Too much fluid in the cavity (such as in a pericardial effusion) can result in pericardial tamponade, and compression of the heart within the pericardial sac which restrict the heart from fully dilating during diastole. The extra fluids in tamponade are usually drained by inserting a needle percutaneously into the pericardial cavity. The pericardial cavity is also the target for insertion and fixation of epicardial electrodes for cardiac pacing; for epicardial ablation treatment; and for delivering medications. Epicardial electrodes are attached (fixed) to the epicardium and can be used for sensing the intrinsic cardiac electrical activity and stimulating (pacing) the heart by delivering electrical impulses from an artificial pacemaker.

A known percutaneous sub-xiphoid pericardial access procedure uses a 17-gauge Tuohy needle (typically ˜100 mm overall length and 1.5 mm outer diameter). As the needle approaches the heart under fluoroscopic guidance, small amounts of contrast media are injected to monitor penetration of the needle tip into the pericardial space. Proper positioning of the needle in the pericardial space is identified by trapping, and a typical expansion, of the contrast media in the pericardial space. Once the space has been located, a guidewire is passed through the needle and the distal end positioned in the pericardial space. An introducer sheath can then be pushed over the wire to form a channel for delivering tools and/or treatments into the pericardial space. However, in practice this technique is not easy. One difficulty in any percutaneous minimal invasive pericardial locating and accessing procedure lies in the fact that in most patients the pericardial cavity is a thin space between two membranes. Hence, as a needle is pushed toward the heart in an attempt to locate the pericardial cavity, it is difficult to determine when the needle tip is actually within the cavity and in many cases the physician continues to push the needle beyond the cavity and into the myocardium. Moreover, the physician usually then pulls and pushes the catheter back and forth while injecting contrast media to try to locate the cavity. Such manipulations may result in cardiac damage and/or complications. Therefore, it is desirable to prevent these inherent risks of puncturing the myocardium and access the pericardial cavity as fast and as precisely as possible.

WO 2005/016157, by RUPP, Heinz et al. discloses a device for puncturing or otherwise manipulating human or animal tissue, which detects and signals when tissue has attached itself to the device. The device allows access to the interior of the pericardium once the pericardium has been pierced. The device can be used in minimally invasive surgery when it is necessary to determine that a tissue or organ has attached to a surgical device such as a puncturing device. The device described in this patent has a deflection mechanism and exhibits a head with a side opening for collecting a punctured tissue. After detection of the organ, e.g., the myocardium, the pericardial sac is sucked by a deflection mechanism (vacuum) to be stabilized to the catheter, thereafter the pericardial sac is pierced in order to enter the pericardial space. A deflecting mechanism requires that the opening at the distal end of the catheter to which the organ is to be attached by deflection needs to be in full and optimal attachment with the desired tissue before beginning of the deflection in order to sufficiently attach the distal end with the desired tissue. Hence, it is not always easy to acquire such optimal attachment and therefore, the attachment may fail.

U.S. Pat. No. 5,972,013, to Schmidt; Cecil C., discloses a device and method for minimally invasive access to the pericardial space. The device includes a penetrating body axially mobile within the lumen of a guide tube. The guide tube includes a deflecting mechanism for deflecting the distal end of the penetrating body. In use, the pericardium is contacted with the distal end of the guide tube and suction is applied to form a pericardial bleb. The penetrating body is axially moved distally within the lumen of the guide tube until the deflecting mechanism deflects the penetrating body to cause the penetrating end of the penetrating body to enter the bleb of pericardial tissue at an angle oblique to the longitudinal axis of the guide tube. This patent suffers from similar limitations as application WO 2005/016157. In addition, the device of U.S. Pat. No. 5,972,013 does not include any means to ensure the location of the desired tissue, e.g., the pericardial sac.

U.S. Pat. No. 5,931,810, to Grabek; James R., discloses an apparatus for creating a bleb of pericardial tissue space. The apparatus includes a shaft with jaws on its distal end a handle on its proximal end for activating the jaws. A needle, for puncturing a bleb of tissue grasped within the jaws, is movably mounted within the shaft bore. This device for locating and accessing the pericardial space does not include means to ensure the location of the desired tissue, e.g., the pericardial sac. In addition, the grasping jaws apparatus may not be sensitive enough to ensure the exclusive grasping of the pericardial sac without the co-grasping of adjacent tissue.

It would be highly advantageous to have a system, apparatus and/or method of performing minimally invasive pericardial procedures, with an improved accuracy and safety level.

### SUMMARY OF THE INVENTION

There is provided, in accordance with an embodiment of the present invention, an apparatus, system, and method for performing minimally invasive pericardial procedures, with an improved accuracy and safety level.

According to some embodiments, devices and methods are provided for performing a treatment inside or proximal to a pericardial space in a minimally invasive fashion. An example of such a device includes a needle having a sensing means to detect substantially different bodily tissues and to enable a practitioner to determine when the distal end of the needle is in the pericardial space; an anchoring means to secure the distal end of the needle in the pericardial space; and a delivery means for delivering a guidewire to the pericardial space to perform the treatment.

In some embodiments the device's needle is at least bifurcated to enable entry of a needle, a guidewire, and an anchoring means to a selected pericardial space.

In some embodiments the device's sensing means is selected from the group consisting of optical sensors, body property detectors, contrast material distributors, electrical activity sensing mechanisms, mechanical sensors, and pressure sensing mechanisms.

In some embodiments the device's sensing means are sensible in real time.

In some embodiments the device's anchoring means is selected from the group consisting of hooks, suction mechanisms, and anchors.

In some embodiments the device's delivery means includes a steering mechanism for determining the direction of the guidewire for delivery to at least one selected location.

In some embodiments the device may further comprise a means for separating a pericardium from a myocardium.

According to one example of such a method, the process includes entering into the body a device adapted for locating a pericardial space, the device including sensing means to detect substantially different bodily tissues; monitoring the sensing means to enable a practitioner to determine when the distal end of a needle is in the pericardial space; and when the indication has been identified, maintaining the device in a selected position relative to the pericardial space, and optionally delivering a guidewire to the pericardial space to perform a treatment. According to some embodiments a method for delivering a guidewire to at least one pericardial location in a minimal invasive way is provided, including: locating a selected location in a pericardial space, using a needle having a sensing means to determine when the distal end of the needle is in the pericardial space; anchoring the needle in the pericardial space and/or to the pericardial sac; and entering a guidewire to at least one selected location in the pericardial space, by guiding the guidewire through the needle.

In some embodiments a device is provided for delivering a guidewire to at least one pericardial location in a minimal invasive way, including: a needle having a sensing means to determine when the distal end of the needle is in the pericardial space; an anchoring means to secure the distal end of the needle in the pericardial space; and a guidewire to be delivered to the selected location in the pericardial space, wherein the guidewire is deliverable through said needle.

In still further embodiments a device and method for treating Bundle branch block is provided, the device including: a means for locating at least one pericardial location using a delivery lumen; a means for anchoring the delivery lumen at least one pericardial location; and a means for delivering at least one electrode to at least one pericardial location, using the locating means.

According to some embodiments, a plurality of electrodes may be delivered to a plurality of pericardial locations; and a plurality of pulses may be generated by the respective electrodes, each said pulses configured to optimally synchronize cardiac electrical and mechanical activity.

According to some embodiments, the present invention provides a system for delivering a guidewire to a pericardial cavity of a heart. The system of the invention has a delivery device with a slender shaft having a proximal end and a distal end.

In one embodiment of the invention, the shaft contains a bi-luminal needle extending from the proximal end to the distal end of the shaft. The distal end of the second lumen extends beyond the distal end of the first lumen by a distance D selected to be substantially equal to the thickness of the myocardium.

In use, the distal end of the second lumen is inserted through the myocardium and into the interior of a heart chamber, such as the left ventricle. The system further comprises anchoring means including an anchor having a low caliber profile and a high caliber profile. After delivery of the distal end of the second lumen into the heart chamber, the anchor in the low caliber profile is delivered through the second needle and advanced into the heart chamber. The anchor is then brought to the large caliber profile and applied to the chamber surface of the endocardium. In this configuration, the distal end of the first lumen is located in the pericardial cavity, due to the fact that the second lumen extends beyond the distal end of the first lumen by the distance D which, as explained above, is selected to be substantially equal to the thickness of the myocardium. The guidewire may then be delivered through the first lumen to the distal end of the first lumen and into the pericardial cavity. The anchor may be made, for example, from a preshaped material such as Nitinol capable of being constrained in the low caliber profile and spontaneously reverting to the high caliber profile upon removal of the constraint. As another example, the anchor may be an inflatable balloon.

Movement of the anchoring means or the guide wire through a needle of the delivery device may be manual, or may make use of a motorized unit.

Other embodiments of the invention comprise a needle and having a sensor detecting a property of body tissue adjacent to the distal end of the needle. The property may be, for example, an optical property, such as color, or a mechanical property such as elastic resistance. These embodiments of the invention take advantage of the fact that the pericardial cavity has different properties, such as optical, mechanical or physical properties, than surrounding tissues, so that the pericardial sac and cavity can be detected.

The system of the invention may further comprise means for expanding the pericardial cavity.

The system of the invention, according to some embodiments, may comprise means for imaging a body region adjacent to the distal end of the needle in order to ascertain that the distal end of the needle is in fact attached to the pericardial sac or located in the pericardial cavity. The system of the invention may comprise means for locally expanding the pericardial cavity separating (locally at the region of the needle tip) the pericardial sac from the epicardial surface. Such means may involve, for example, fluid injection, such as saline, from a distal tip of one of the needles. Other means for expanding the pericardial cavity may include a balloon configured to be inflated in the pericardial cavity, or use of suction or hooks to pull the parietal pericardium from the visceral pericardium.

After the guidewire has been delivered to the pericardial cavity, the delivery device may be removed while leaving the guidewire in place. A surgical or therapeutic device may then be delivered to the pericardial cavity along the guide wire.

In one embodiment, the delivery device of the invention comprises a motorized unit for delivering an anchoring device and the guidewire through the needles of the delivery device. The motorized unit may be integral with the delivery device or may be reversible attachable to the device.

The device and method of the invention may be used as preparation for the introduction into the pericardial space of surgical tools such as a catheter for navigation, fixation of epicardial electrodes, an epicardial ablation catheter, endoscope or other tools. The invention may also be used to introduce medicine into the pericardial cavity.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 illustrates a front view (FIG. 1A), a top view (FIG. 1B) and a longitudinal section (FIG. 1 C) of a device for delivering a guidewire to a pericardial space according to one embodiment of the invention;

FIG. 2 shows a cannula configured to enable selectable bending at the distal end mounted on a guide wire, according to some embodiments;

FIGS. 3A to 3D illustrate delivery of a guide wire to the pericardial cavity, according to some embodiments;

FIG. 4 Illustrates a device for delivering a guide wire to the pericardial space in accordance with another embodiment of the invention;

FIGS. 5A to 5D illustrate delivery of a guide wire to the pericardial cavity using the device of FIG. 4;

FIG. 6 illustrates a motorized unit for use with the guidewire delivery device, according to some embodiments;

FIG. 7 shows a device for delivering a guidewire to a pericardial space according to an embodiment of the invention having an optical sensor;

FIGS. 8 and 9 show use of the device of FIG. 7 for locating a pericardial space, according to some embodiments;

FIG. 10 shows a device for delivering a guidewire to a pericardial space according to an embodiment of the invention having a mechanical sensor comprising a pressurized fluid;

FIGS. 11 and 12 show use of the device of FIG. 10 for locating a pericardial space, according to some embodiments;

FIG. 13 shows a device for delivering a guidewire to a pericardial space according to an embodiment of the invention having a mechanical sensor comprising a spring biased rod;

FIG. 14 shows a device configured to separate the pericardial sac from the epicardium using hooks, according to some embodiments;

FIG. 15 shows a device configured to apply suction to separate the pericardial sac from the epicardium, according to some embodiments; and

FIG. 16 shows a delivery assist device for delivering a device for locating a pericardial cavity, according to some embodiments;

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

The word “needle” as used herein may refer to a variety of lumen, cannulas, stylets, trocars or other instruments used to inserting into the vein, artery, or body cavity or space. The term “pericardial space” may refer to one or more selected locations in or substantially adjacent to the pericardial space and/or the pericardial sac.

Embodiments of the present invention include apparatus', systems, devices and methods for performing minimally invasive pericardial procedures, with an improved accuracy and safety level. According to some embodiments of the present invention, a cannula or needle may be safely inserted into a patient through the epicardium and the myocardium into a cardiac chamber cavity. Following the positioning of the lumen in the pericardial space, the lumen may be used to hook onto or otherwise attach to the pericardial sac without puncturing or harming the myocardium, in order to separate the pericardial sac from the myocardium, and thereby generate a space between the sac and the myocardium (epicardium) to where a guidewire may be safely delivered and/or treatment may be implemented.

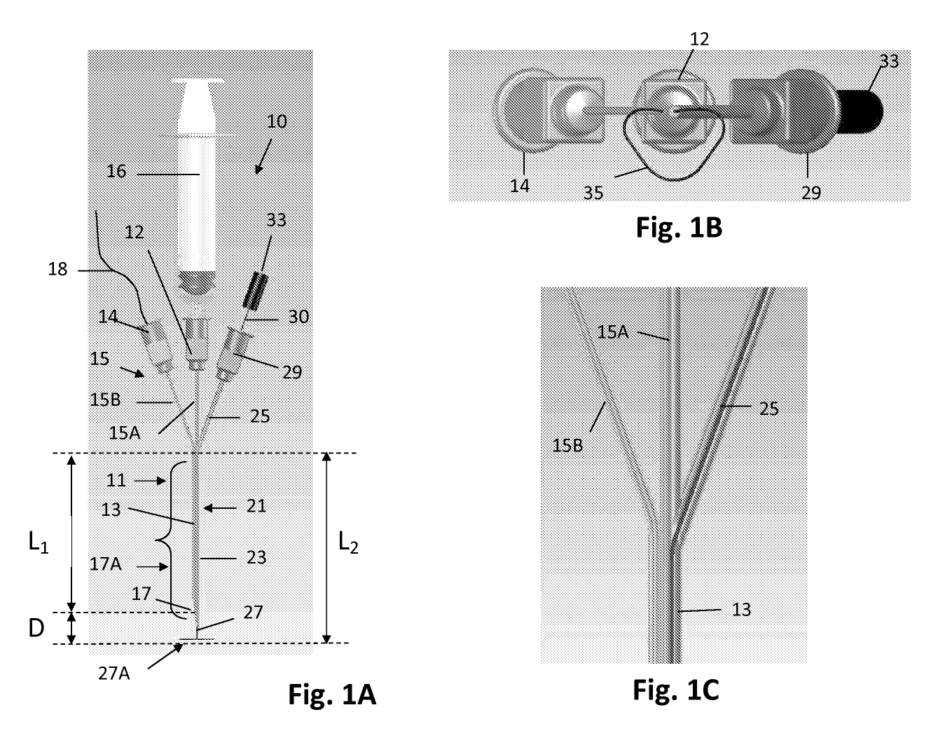

Reference is now made to FIGS. 1A to 1C, which illustrate a device 10 for detecting substantially different bodily tissues, thereby enabling a practitioner to determine when the distal end of the device is in or proximate to the pericardial space, and optionally delivering a guidewire to a pericardial cavity (PC) of the heart to perform a selected treatment, in accordance with some embodiments of the present invention. The device 10 comprises a needle with a main lumen 11. A second lumen 21 is attached to the main lumen 11 and parallel to it. The main lumen 11 comprises a penetrating portion 13 of length L1 and a port portion 15, and terminates in a tip 17, for example a beveled tip. The main lumen 11 is bifurcated at the junction between the penetrating and port portions so that the port portion 15 comprises at least two sub-portions 15A and 15B. The bifurcation is shown in greater detail in the longitudinal section shown in FIG. 1C. The sub-portion 15A is connected to a syringe port 12 and the sub-portion 15B is connected to a guidewire port 14. The port 12 is adapted to be connected to a syringe 16, typically with a Luer lock fitting. As explained below, the main lumen 11 is adapted to receive a guidewire 18 extending from the guidewire port 14 to the tip 17.

The second lumen 21 comprises a penetrating portion 23 and a port portion 25. The penetrating portion 23 terminates distally in a penetrating tip 27. The penetrating portion 23 has a length L2>L1, satisfying D=L2−L1, where D is the distance that the penetrating portion 23 of the second lumen 21 extends beyond the distal tip 17. The port portion 25 terminates proximally in an anchoring wire port 29. The second lumen 21 is adapted to receive an anchoring wire 30 extending from the port 29 to the tip 27.

The device 10 is inserted by means of an introducer through the epicardium and the myocardium into a cardiac chamber cavity until the penetrating tip 27 of the second lumen 21 enters the chamber cavity.

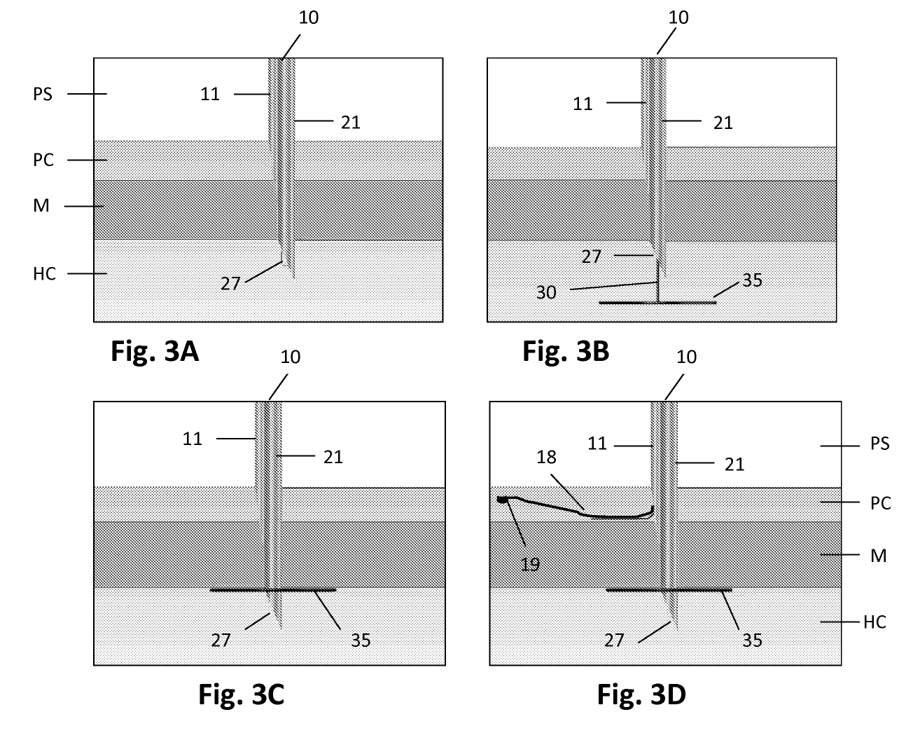

FIGS. 3A to 3D shows use of the device 10 for accessing the PC of a heart, which is located between the pericardial sac (PS) and the myocardium (M). As shown in FIG. 3A, the distal end of the device 10 is first introduced percutaneously through the pericardial sac PS, the PC, and the myocardium M and into a heart chamber HC such as the left ventricle cavity (FIG. 3A). The presence of blood in the anchoring wire port 29 may serve as an indication that the tip 27 of the second lumen 21 has penetrated into the heart chamber HC. Therefore, a cap (not shown) may be used to cover the anchoring wire port 29 to prevent blood from leaking out of it. Such a cap may be substantially transparent to allow for viewing of blood or other fluids entering the cap. Penetration of the heart chamber HC may be also detected by imaging means, for example, X-ray, Doppler or other means.

The anchoring wire 30 is pushed through the lumen of the second lumen 27 into the heart chamber HC, either manually using a grasping handle 33 (FIGS. 1A and 1B) attached to the proximal end of the anchoring wire 30, or by a motorized delivery system, described below. The distal end of the anchoring wire 30 may be pretreated to endow it with shape memory. The distal end of the anchoring wire is constrained in its low caliber profile when inside the distal end 27, and spontaneously reverts to a high caliber profile when extended beyond the distal end of the lumen 21 (also shown in FIG. 1B). Thus, when the distal end of the anchoring wire 30 extends beyond the distal end 27 of the second lumen 21 to the endocardial space, the anchor 35 emerges into the heart chamber HC and adopts its large caliber profile. (FIG. 3B) After release of the anchor 35 in the endocardial space, the anchoring wire may be pulled back in a proximal direction to apply the anchor 35 to the distal end 27 of the lumen 21. (FIG. 1C). Anchoring device 35 may have one or more selected materials, forms, sizes and/or shapes to enable effective anchoring of lumen 27 to a target location. The device 10 may be pulled back proximally together with the anchoring wire, until the anchor 35 is applied to the endomyocardium wall M, thereby preventing further proximal movement of the device 10. In this position the distal end 17 of the main lumen 11 at least partially opens into the PC, since the distance D is selected to be about equal to the thickness of the myocardium M. In another embodiment of the bilumen needle, the distance D can be modified by sliding the main lumen 11 along the second lumen 21 as far as required. Prior to insertion of a guidewire through the needle 11, the pericardial space PC may be expanded by delivering though the needle 11 a fluid such as sterile saline or nitrogen gas to inflate the pericardial space. This may be done, for example, to facilitate the insertion of a guidewire 18 into the PC or to enhance maneuverability of a surgical tool in the pericardial space after delivery of the tool to the pericardial space.

The guidewire 18 may then be inserted through the guidewire port 14, into the main lumen 11 if it was not inserted previously and pushed distally until it exits the tip of the main lumen 11 into the pericardial cavity (PC). The guidewire 18 may be, for example, a 0.014-0.038 inch guidewire, or may be of other relevant sizes. The tip of the guidewire may be provided with padding 19 in order to reduce or prevent damage to the myocardium M or the pericardial sac. In some embodiments the tip of the guide wire is curved, and in some embodiments the tip may have resilient flexibility. As the tip of the guidewire passes through the lumen of the main needle 11, in some examples, it may be constrained into an essentially straight configuration. As the tip of the guide wire exits the distal end 17 of the main needle 11, it spontaneously adopts the curve configuration as shown in FIG. 3D. Ascertaining that the guidewire 18 has in fact entered the PC may be performed by injection of a contrast agent from the syringe 16 through the additional port 12 and imaging the guidewire 18, or by using other imaging methods. When the tip 17 of the needle 11 is located within the PC the contrast agent is substantially trapped in the pericardial cavity and delineates the contour of the heart.

Once it has been ascertained that the guidewire 18 is located within the PC, the anchor wire is retracted into the lumen 21 and is constrained in its low caliber profile. The device 10 is then removed together with the anchor wire, leaving the guidewire 18 in place with its tip located within the PC. A surgical tool or other relevant device or material may then be delivered along or through the guidewire 18 to the pericardial cavity.

In another embodiment of the invention, the device 10 comprises a single bifurcated needle that serves for the delivery of both a guide wire and an anchoring wire.

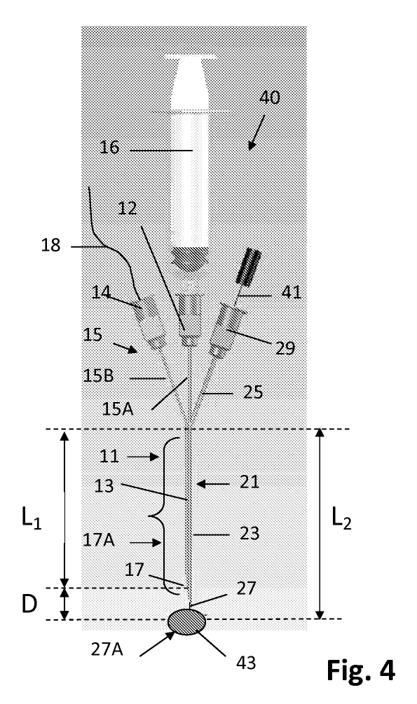

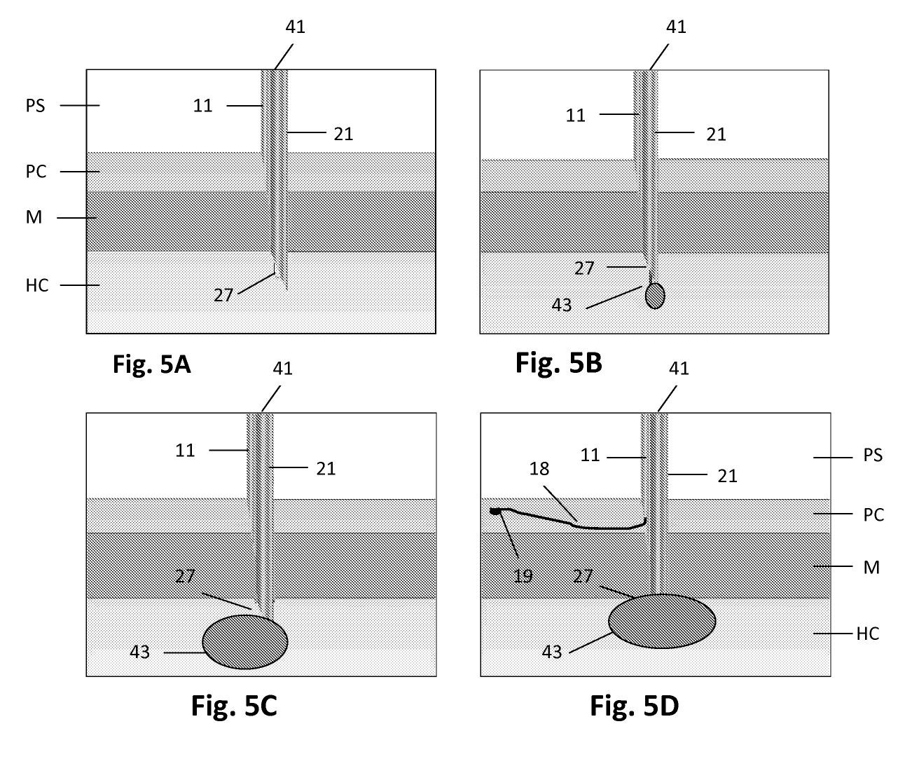

According to some embodiments, as can be seen with reference to FIG. 4, a device 40 (elements of which correspond to the elements of the device 10, and are indicated by the same reference numerals without further comment) may include a balloon catheter 41 mounted within the lumen 21. Balloon catheter 41 may have an inflatable balloon 43 at its distal end that is used to anchor the distal end of the device 40 in the pericardial cavity, instead of or in addition to the anchoring wire 30 used in the device 10. As shown in FIG. 5A, the device 40 is inserted through the pericardial sac PS, PC and the myocardium M into a heart chamber, such as the left ventricle cavity (LV), until the penetrating tip 27 of the second lumen 21 has entered the LV. The distal end of the balloon catheter 41 may be pushed through the lumen 21 until the balloon emerges from the distal tip 27 and enters the Heart chamber HC, as shown in FIG. 5B. Pressurized fluid, such as saline, may then be delivered from the proximal end of the catheter 41 through the lumen of the catheter shaft into the balloon 43 to inflate the balloon inside the ventricular cavity (FIG. 5C). After inflation of the balloon 43, the shaft of the balloon catheter 41 is pulled back in a proximal direction to apply the balloon to the myocardium M and prevent further proximal movement of the device 40. As with the device 10, in this position, the distal end 17 of the main lumen 11 at least partially opens into the PC, since the distance D is selected to be about equal to the thickness of the myocardium M. In another embodiment of the bitumen needle, the distance D can be modified by sliding the main lumen 11 relatively along the second lumen 21. The guidewire 18 is then inserted through the guidewire port 14 into the main needle 11 and pushed distally until it exits the tip of the main needle 11 into the PC (FIG. 5D). In some embodiments, a larger than required portion or length of guidewire may be pushed into the PC (e.g., more guidewire distance than is strictly required to reach inside the PC) such that when the delivery device is extracted, the operator may be certain that at least some of the guidewire may remain within the PC.

Ascertaining that the guidewire 18 has in fact entered the PC may be performed by injection of a contrast agent from the syringe 16 through the additional port 12 and imaging the guidewire 18, or by using other imaging methods. When the tip 17 of the needle 11 is located within the PC the contrast agent is substantially trapped in the pericardial cavity and delineates the contour of the heart. The balloon is then deflated to collapse it to its lower caliber profile, and is drawn back into the lumen 21. The device 40 is then pulled out, leaving the guidewire distal tip within the pericardial cavity. Active measures may be taken to ascertain that adequate portion of the distal end of the guide wire remain within the pericardial cavity. For example, while an operator may extract or pull the device out of the patient's body, s/he may hold the guide wire with contra force and even push the guidewire further into the PC, thereby ensuring that the guidewire remains within the PC when extracting the delivery device. Once it has been ascertained that the guidewire 18 is located within the PC, the device 40 is removed leaving the guidewire 18 in place with its tip located within the PC. Other tools may then be delivered along the guidewire 18 to the pericardial cavity.

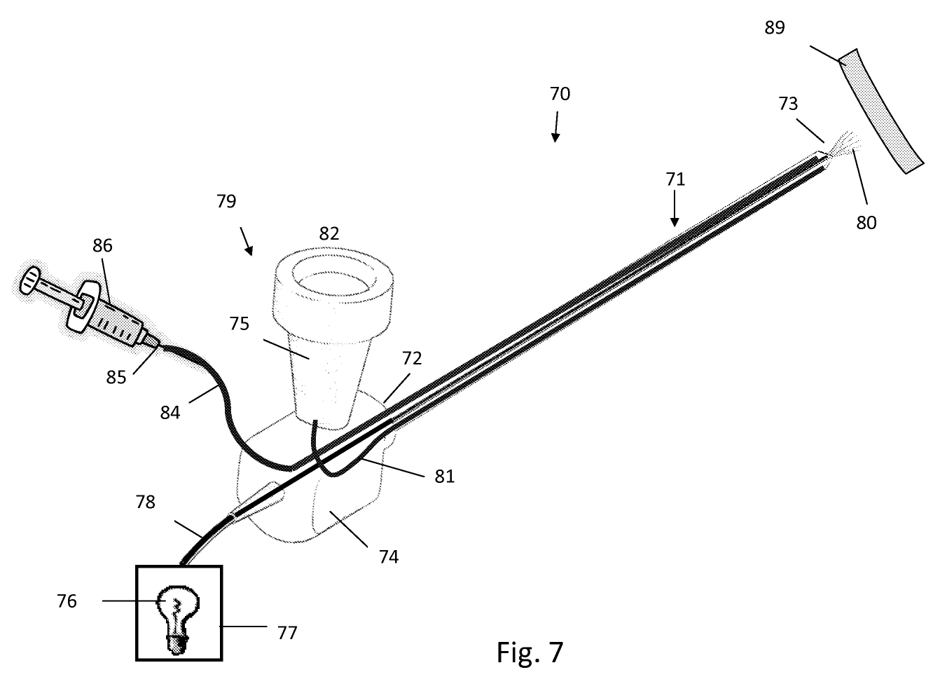

Reference is now made to FIG. 7, which shows a device 70 for locating a PC in accordance with a second embodiment that takes advantage of the fact that the pericardial sac has a typical off-white color while the tissues located externally to the sac and the myocardium located internally within, are typically darker. The device 70 comprises a needle 71 having a proximal end 72 and a distal end 73. The distal end 73 terminates in a sharp tip. The proximal end 72 is attached to a viewing unit 79 having a body 74 and an eyepiece 75. The device 70 further comprises a light source 76 contained within a housing 77. A first light guide 78 extends from the light source 76 through the viewing unit body 74 and the needle 71 to the distal end 73. In use, light 80 emitted from the distal end 73 is reflected from body tissue 83 and is collected by a second optical fiber 81 extending along the needle 71. The proximal end of the optic fiber 81 is optically coupled to an ocular lens 82 which focuses light 80 reflected from tissue for viewing. The shaft 71 is surrounded by an opaque coating to prevent stray light from exerting the optic fibers.

The device 70 may also be provided with a rinsing system for rinsing the distal end 73 from blood and other tissue debris which may adhere to the distal end 73 and which might otherwise partially or completely block the incident illumination or the reflected light. The rinsing system may also be used for draining fluids from distal end 73 or for injecting agents such as contrast media from the distal end 73 of the needle 71. The rinsing system may include, for example, a small diameter tube 84 extending along the shaft or as a lumen within the device 70. The tube 84 terminates at its proximal end with a Luer lock fitting 85, to which a syringe 86 containing a fluid, such as sterile saline may be attached. The fluid is conducted from the syringe to the distal end of needle 71 and is released from the distal end 73 so as to rinse the distal end of the needle. Release of the fluid from the syringe can be performed manually or using a motorized device. The needle 71 may contain one or more additional lumens (not shown), for example, for various other functions. Light collected by the optic fiber 81 and conducted to the viewing unit 74 may be conducted to a transducer (not shown) which generates an electrical signal that can be digitized and analyzed to give an indication for the color and nature of the tissue located at any time adjacent to the distal end 73 of the needle 71. The transducer may be a CCD camera which continuously acquires the light that is conducted by the optic fiber 81. The results of the analysis may be displayed on a screen.

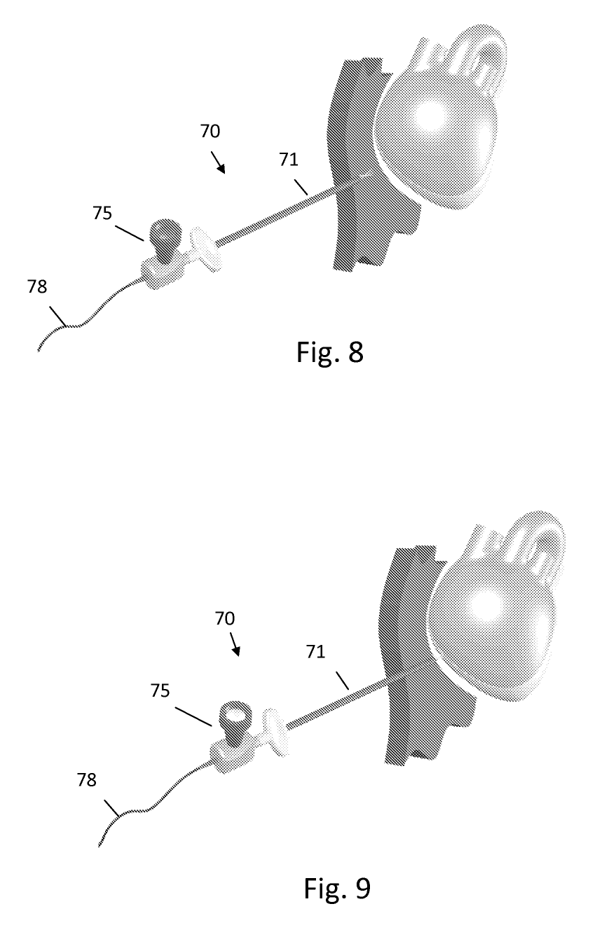

As shown in FIG. 8, in use, the distal end of the shaft 71 is advanced in the body towards the heart, as explained above with reference to the first embodiment. As the distal end of the shaft advances from the skin towards the heart, light collected by the light guide 81 is continuously monitored and changes in the color of the tissue adjacent to the distal end is observed or analyzed. The distal end of the shaft is adjacent to the pericardial sac when the off-white color of the sac is observed (FIG. 9). The distal end of the needle 71 can then be advanced to puncture the sac and penetrate the pericardial space. A guidewire can then be delivered through the lumen of the needle into the space.

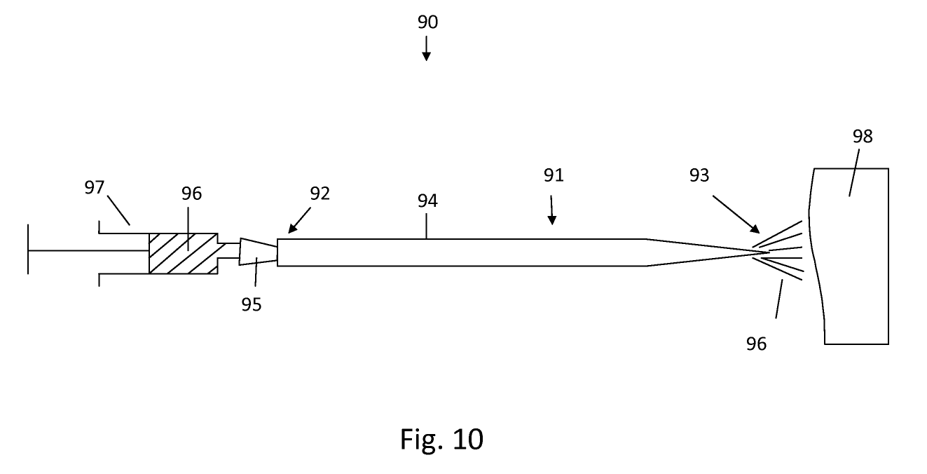

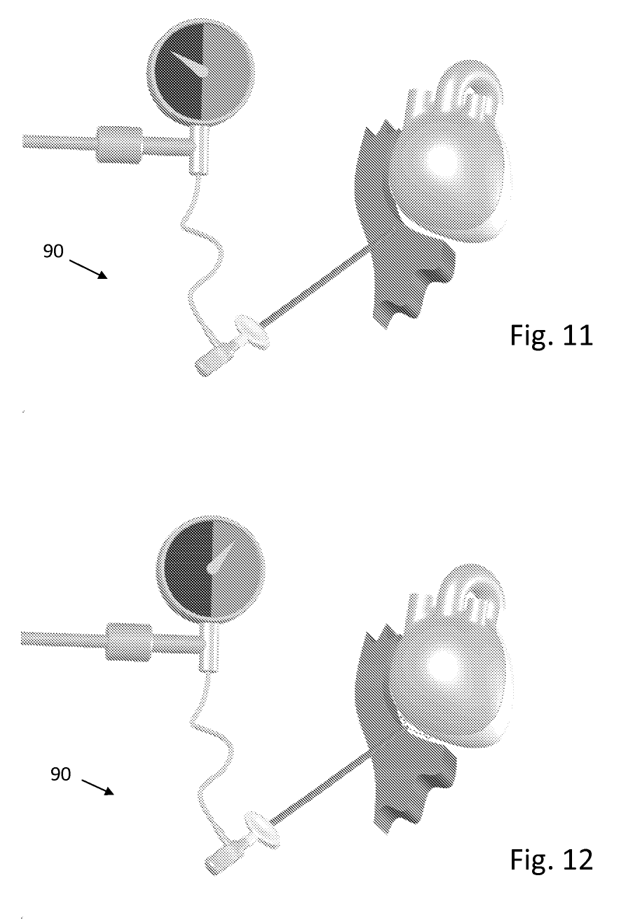

According to some embodiments, as can be seen with reference to FIG. 10, a device 90 may be used for locating the pericardial cavity. The device 90 utilizes variations in the mechanical properties of tissues in the vicinity of the pericardial cavity. The device 90 comprises a needle 91 having a proximal end 92 and a distal end 93. A channel 94 extends through the needle 91. The proximal end of the shaft 91 terminates in a port 95 that is adapted to be attached to a source 97 of a pressurized fluid 96. The pressurized fluid may be a liquid or gas and the source 97 may be a syringe attached to the port 95 as shown in FIG. 10. The pressurized fluid is delivered though the channel 94 and is released at the proximal end 93 of the needle 91. The fluid 96 is maintained at a constant pressure that is greater than atmospheric pressure. The flow rate release of the fluid from the distal end 93 of the needle 91 is determined by the resistance encountered by the dense of the density properties of tissue 98 that surround the needle tip 93 During the percutaneous sub-xiphoid procedure, the needle distal tip is pushed and meets different tissues as it advances toward the heart. The various tissues comprise specific tissue densities and present a different resistance to the flow. When the distal end 93 is located in a relatively dense tissue, the resistance at the tip of the needle is relatively high, so that the flow rate (the volume of fluid that flows out from the needle per unit time) is low (FIG. 11) When the needle tip has penetrated into the pericardial cavity, the resistance at the needle tip decreases and the flow rate is consequently relatively high (FIG. 12). The change in flow rate and/or the pressure of the fluid within the needle detected when the distal end of the needle enters the pericardial cavity is an indication that the distal end has in fact penetrated into the pericardial cavity. In addition, the fluid in the needle can be a contrast media which can be monitored by fluoroscopy. Hence, the location of the pericardial cavity can simultaneously be detected by observing the characteristic spread of the contrast media over the contour of the heart.

Another option for maintaining fluid pressure within the needle is a constant flow syringe apparatus, for example, as disclosed in http://hplc.chem.shu.edu/NEW/HPLC_Book/Instrumentation/pmp_cflw.html and http://www.warneronline.com/product_info.cfm?name=DN%20Series%20Constant%20Flow%20Syringes&id=772. The fluid flow rate and/or the fluid pressure can be monitored by several means. For example, if the fluid 96 is a contrast media, the flow at the distal end 93 can be observed by fluoroscopy. The flow rate and/or the fluid pressure can also be analyzed by computing means and displayed on a monitor. The flow detection can also use an optical device. Another embodiment uses a pressure detector that monitors the pressure of the fluid within the syringe or within the needle. The needle 91 may contain one or more additional lumens (not shown), for example, for various other functions.

When it has been determined that the distal end 93 is positioned in the pericardial cavity, the source 97 may be detached from the port 95, and a guide wire (not shown) inserted into the port 95, if it was not inserted previously, and delivered to the distal end 93 and into the PC.

At any time during one or more of the above described procedures the operator may withdraw the plunger of the syringe in order to draw into the syringe any fluids in the vicinity of the distal end of the needle, for example, in order to inspect body fluids at the tip of the needle.

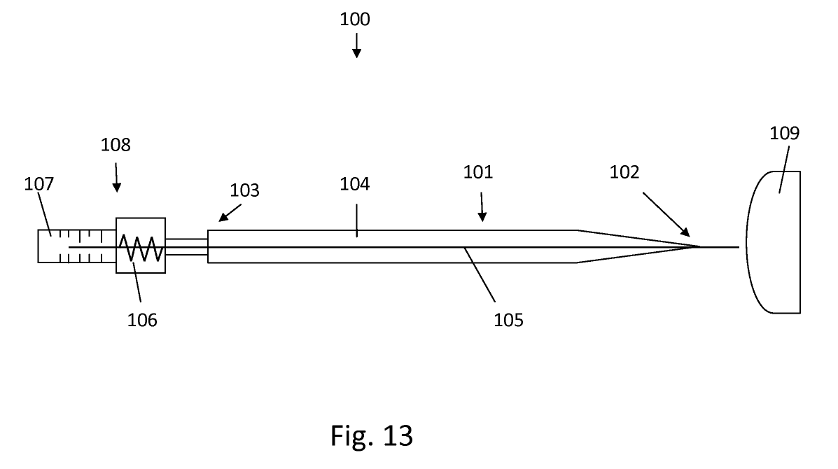

Reference is now made to FIG. 13, which shows a device 100 for locating the pericardial cavity, in accordance with other embodiments of the invention. The device 100 comprises a needle 101 having a distal end 102 and a proximal end 103. A rigid rod 105 extends along the lumen of the needle 104 and extends beyond the distal end of the needle. The device 100 further comprises a handle 108. The rod 105 extends into the handle 108. The rod is attached to a spring 106 in the handle 108, so that the rod is spring biased in a resting position shown in FIG. 13. The device 100 detects axial motion of the rod 105 by means of a scale 107 associated with the handle 108. When the distal end 102 of the needle is located near the heart 109, motion of the heart systole and diastole cycle is transferred to the rod 105 which oscillates axially in the needle 101. Oscillation of the rod 105 is observed as an oscillation of the proximal end of the rod 105 against the scale 107. The needle 101 may contain one or more additional lumens (not shown), for example, an optic fiber, a lumen for delivering contrast media, a guidewire, or other elements.

As the needle 101 is advanced towards the heart, the proximity of the distal end of the needle to the heart is determined by any one or more of the amplitude, speed and the acceleration of the oscillations of the rod. The motion of the rod can also coupled to a transducer (not shown) which may convert the rod movements into an electric signal that may be analyzed to determine the properties of the oscillations and the position of the distal end of the needle relative to the heart. The results of the analysis may be displayed on a screen. In another embodiment, a hydraulic piston is used instead of the spring 106 to bias the rod in its resting position. In this case, movement of the rod can be monitored by monitoring the pressure in the piston or the magnitude of the piston movements.

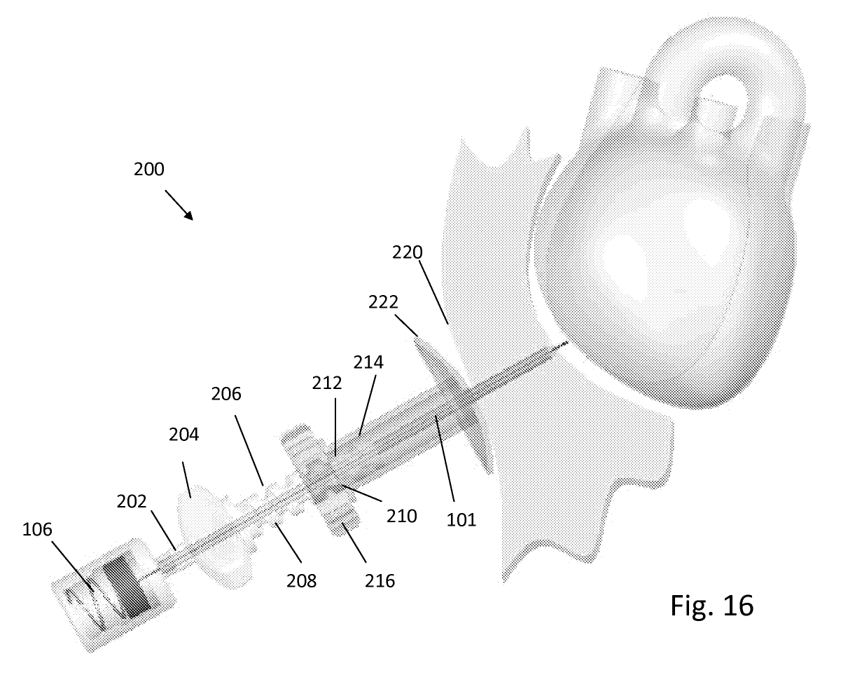

Any of the embodiments of the invention may be used with a delivery assist device for controlled insertion of the needle. FIG. 16 shows, as an example, the device 100 of FIG. 13 mounted onto a delivery assist device 200. With the delivery assist device, advancing the needle 101 inside the body is performed using a mechanical system, instead of pushing the needle by hand through the body. The mechanical delivery can use a motorized or a manual mechanism. The delivery device allows setting and controlling the direction of needle penetration as well as the penetration rate. Normally, in percutaneous pericardial cavity access the pericardium is distanced several cm in a straight line from the point of needle insertion into the body. The delivery device 200 has a hollow shaft 202 upon which the pericardial locating device, such as the pericardial locating device 100, is mounted. The device 200 is stabilized on the body surface 220 by a shield 222. On the shaft, a handle 204 and a cylinder 206 having outer screw threads 208 are mounted. The outer screw threads 208 on the cylinder 206 mate with inner screw threads 210 on an annulus 212 mounted inside a hollow cylinder 214. Rotation of a knob 216 causes the cylinder and the shaft to move axially relative to the hollow cylinder. The direction of movement of the shaft is determined by the direction of rotation of the knob.

With the delivery assist device, an operator can set a scheme of advancing the needle at a first speed during the first few cm and reducing the speed as the distal end approaches the region of the pericardial cavity. Combining the detection mode with the pattern of advancing the needle can enhance the accuracy of detecting the pericardial cavity. The needle may be advanced in small increments. The distance the needle has been advanced at every step is a parameter that can be set by the operator. At every step, the operator can use the detection component to evaluate the needle tip location. If when using the detection component (and optionally fluoroscopy) the operator sees that the needle tip has not yet reached the pericardium, s/he can advance the needle another step. This mode of operation enhances the control, accuracy and safety of the process of detecting the pericardial sac and cavity while refraining from injuring and/or penetrating the myocardium.

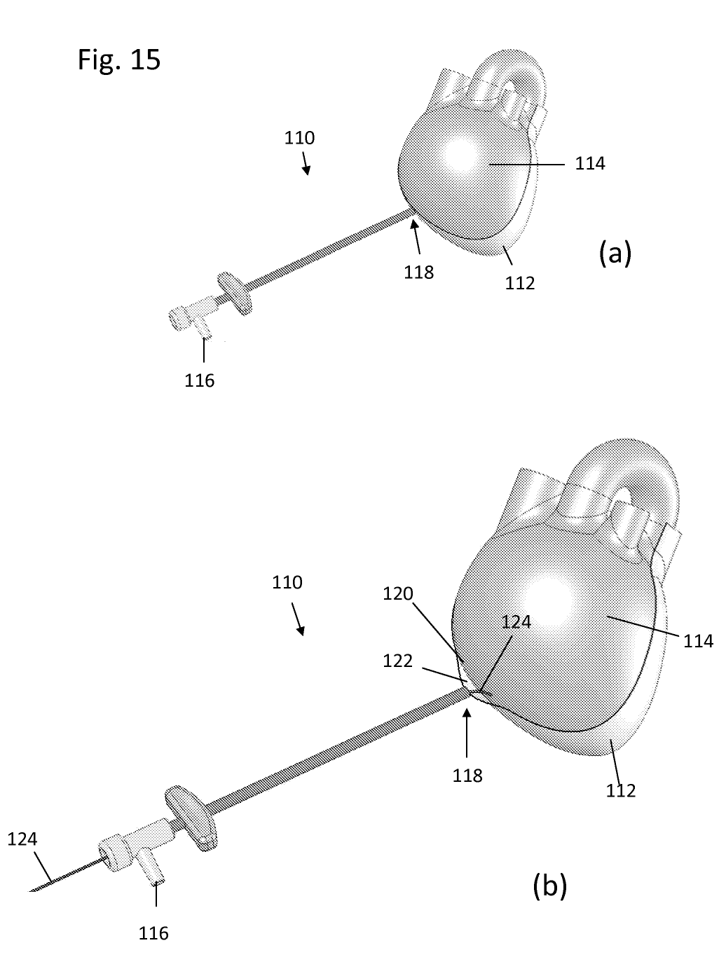

Any of the embodiments of the invention may comprise a lumen for applying suction at the distal end of the device. FIG. 15 shows a needle 110 of any one of the above described devices or embodiments of the invention adapted for applying suction to the pericardial sac 112 of a heart 114 to separate the pericardial sac PS from the epicardium to locally expand the pericardial space. The device may be connected to a source of negative pressure (not shown) by means of a hose 116. As shown in FIG. 15a, the distal end 118 of the needle 110 may be applied to the pericardial sac, and the suction subsequently applied to the pericardial sac. Then, as shown in FIG. 15b, the needle 110 is slightly retracted to pull the pericardial sac 118 from the epicardium 120 in order to locally expand the pericardial space 122. A guide wire 124 may then be delivered to the pericardial space 122, as explained above in reference to the various embodiments of the invention. Once a guidewire has been introduced into the cavity, the suction can be closed and the needle pulled out while maintaining the guidewire in its position.

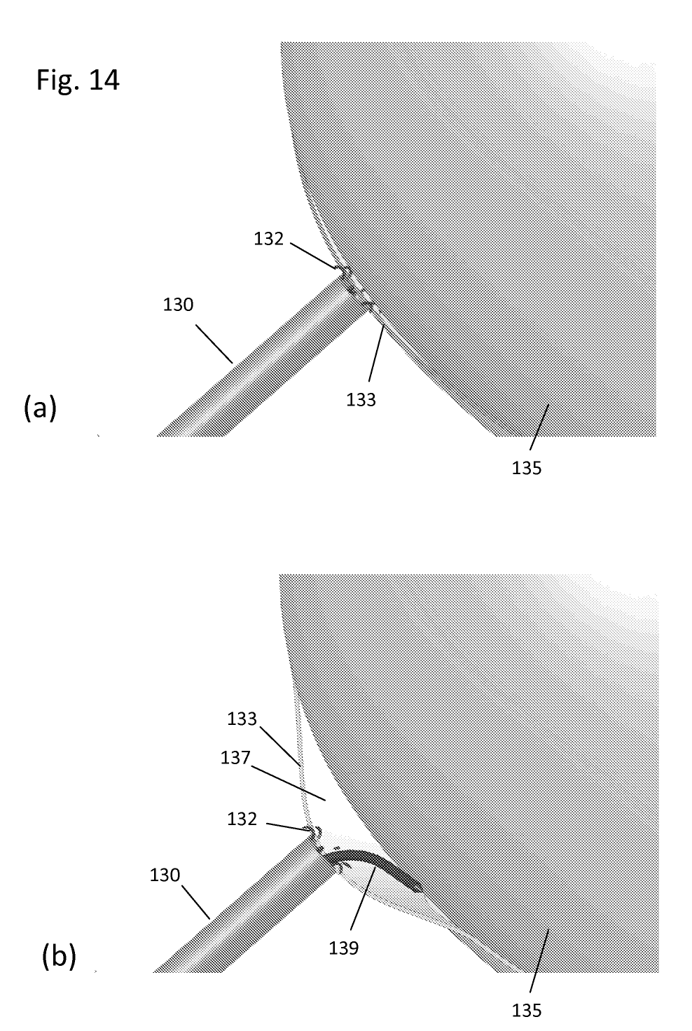

The device of the invention, according to any of its embodiments may comprise a lumen containing one or more hooking wires whose distal ends were pretreated to be a shape memory material. The hooking wires may be made, for example, from Nitinol or other shape memory materials. As shown in FIG. 14a, once the pericardial sac has been located, and the distal tip of the needle 130 positioned on the pericardial sac, the hooking wires are grasped at the proximal end of the needle and advanced in a lumen of the needle, until the distal portion of one or more hooking wires 132 emerge from the distal end of the needle and spontaneously adopt a curved or hooked shape. Hooking wires 132 may have similar or non-similar shapes and/or sizes so as to optimally hook the pericardial sac 133. As the wires emerge, the wires puncture and hook onto the pericardial sac 133 which thus becomes attached to the distal end of the needle, as shown in FIG. 14b. Pulling the needle backwards after inserting the hooks results in pulling the pericardial sac away from the epicardium 135 thus increasing locally the space 137 between the pericardial sac and the epicardium. A guidewire 139 can then be pushed through the needle into the pericardial cavity space as explained above in reference to the use of suction. Once a guidewire or other tool has been introduced into the pericardial cavity, the operator retracts the wires into the lumen of the needle and thus constrains the distal ends of the hooking wires in a straight confirmation.

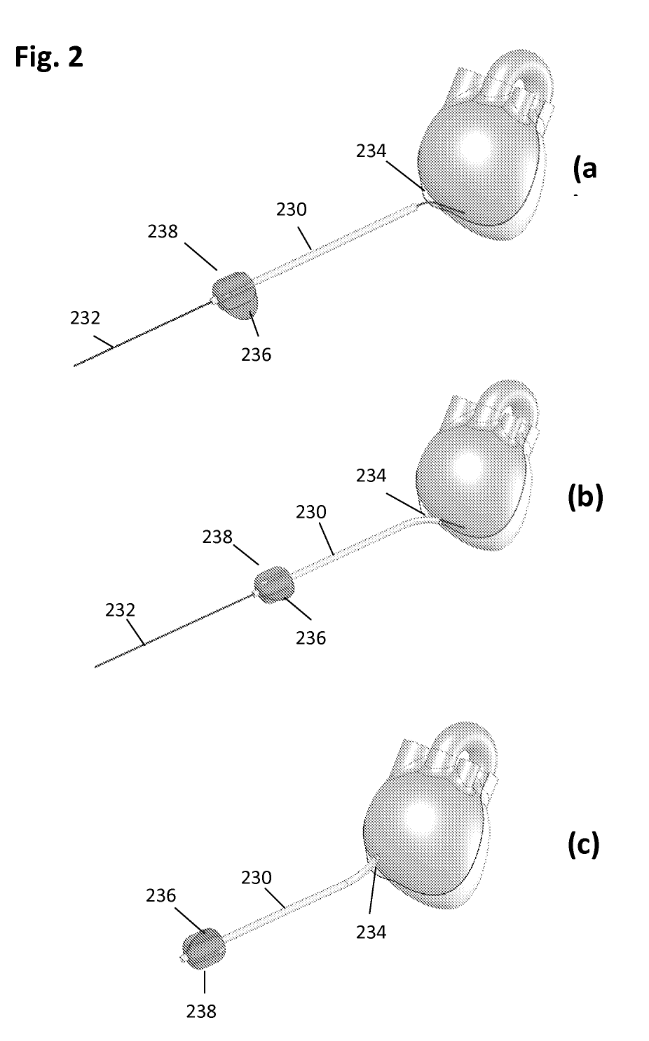

Once a guide wire has been introduced into the pericardial cavity using one or more of the embodiments of the invention, a cannula or sheath may be inserted over the guide wire into the pericardial cavity. FIG. 2 shows a cannula 230 mounted over a guidewire 232 and inserted into a pericardial cavity 234, according to some embodiments. The distal portion of the cannula may be capable of bending in a particular direction (FIG. 2a) relative to the shaft of the cannula. The direction of bending is indicated by markings 236 on a knob 238 located at the proximal end of the cannula so that the direction of bending can be monitored. As the distal end of the portion of the cannula enters the pericardial cavity, the distal end of the cannula encounters the myocardium and bends within the pericardial sac. The shaft of the cannula can then be rotated, for example, by manipulating a knob that is attached to the proximal end of the cannula, so that the bend of the cannula points towards a region of interest within the PC (FIG. 2c) to which a device, such as a catheter, is to be delivered. The catheter is then introduced into a lumen of the cannula and directed to the region of interest by the cannula. This driving mechanism (rotation mechanism) attached to sheath may be used to rotate and direct the distal end of the sheath so as to allow a practitioner to navigate a treatment mechanism in a selected direction and/or to a selected position within the pericardial cavity.

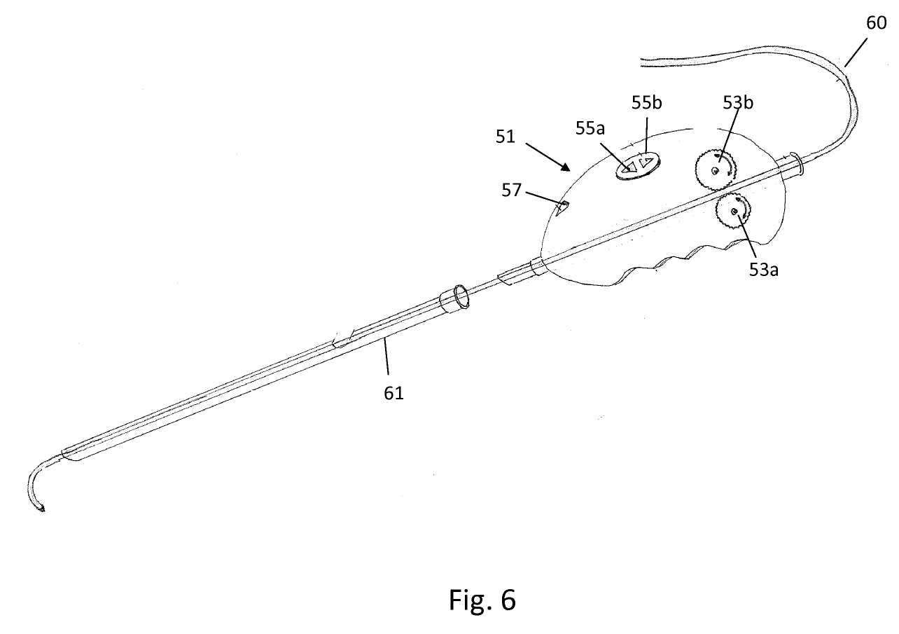

FIG. 6 shows a unit 51 that may be used for inserting or removing a component 60 through a lumen 61 of the device of the invention, such as the device 10 or the device 40. The lumen 61 may be either the main lumen 11 or the anchoring lumen 21. The component 60 may be, for example, the anchoring wire 30, the guidewire 18, the balloon catheter 41, or another suitable inserting component.

The unit 51 may be mounted directly onto the needle 61, as shown in FIG. 6, or may be separated from it. The unit 51 has a housing 57 which is shown in ghost lines to reveal the components of the unit. A component, such as a guidewire is inserted between wheels 53a and 53b. The wheels 53a and 53b are spaced apart by an adjustable distance selected to firmly grasp the component 60 guidewire inserted between them. Due to a sufficiently high coefficient of static friction between the wheels 53a and 53b and the component 60, as the wheels 53a and 53b rotate in opposite directions, the component 60 is translocated in the lumen 61. The wheels 53a and 53b may be rotated manually. Alternatively, the unit 51 may comprise a motor which may be battery operated. The motor may include a mechanism of driving the wheels 53a and 53b in opposite directions. The unit 51 may have a switch, or switches such as 55a and 55b, for selecting the direction of rotation of the wheels 53a and 53, and optionally for speed selection. In another embodiment, the motorized unit 51 may comprise a mechanism other then wheels for advancing the component 60.

The embodiments of the invention may be fabricated to be for single or multiple use. In some examples the delivery elements may be fabricated for substantially single use, while the control mechanism, such as unit 51, may be fabricated for multiple use. In some cases multi-use components may be re-sterilized after use, for example, using ethylene oxide, in order to allow safe reusage. Other combinations may be used.

In any of the embodiments of the invention, the needle of the location or delivery device may also function as an electrode. In this case, the distal tip of the needle serves as an electrode which can be used to acquire the cardiac electrocardiogram signal or other data at the location of the needle distal tip.

According to some embodiments of the present invention, a location and delivery device may be used to accurately deliver one or more electrodes to one or more selective locations in the PC. For example, once the PC has been located a flexible catheter with steering capability is entered into the pericardial space and is maneuvered (navigated) to a selected internal location. Such catheter can carry or provide the lumen through which tools, e.g., electrodes, may be entered through or attached to the primary catheter to one or more selected locations. In some embodiments multiple electrodes may be managed, optionally using external or internal programming, to provide their respective pulses according to an optimal pacing scheme, to optimally synchronize cardiac electrical and mechanical activity.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1-19. (canceled)

20. A device for performing a treatment in a minimally invasive fashion in a pericardial space, comprising: a needle having a sensing means to detect substantially different bodily tissues to enable a practitioner to determine when the distal end of said needle is in said pericardial space; an anchoring means to secure said distal end of said needle inside or in proximity to said pericardial space; and a delivery means for delivering a guidewire to said pericardial space to perform the treatment.

21. The device of claim 20, wherein said needle is at least bifurcated to enable entry of a needle, a guidewire, and an anchoring means to said pericardial space.

22. The device of claim 20, wherein said sensing means is selected from the group consisting of optical sensors, body property detectors, contrast material distributors, electrical activity sensing mechanisms, mechanical sensors, and pressure sensing mechanisms.

23. The device of claim 20, wherein said sensing means is sensible in real time.

24. The device of claim 20, wherein said anchoring means is selected from the group consisting of hooks, suction mechanisms, and anchors.

25. The device of claim 20, wherein said delivery means includes a steering mechanism for determining the direction of said guidewire for delivery to at least one selected location.

26. The device of claim 20, further comprising a means for separating the pericardial sac from a myocardium.

27. The device of claim 26, further comprising a means for delivering a guidewire into a space between said pericardial sac and said myocardium.

28. The device of claim 20, further adapted to perform a treatment in a pericardial space, the device comprising a means to maintain said device in a selected position relative to said pericardial space, while said guidewire is delivered to said pericardial space to perform the treatment.

29. The device of claim 28, wherein said sensing means is selected from the group consisting of optical sensors, body property detectors, contrast material distributors, electrode sensing mechanisms, needle sharpness control mechanism, mechanical sensors, and pressure sensing mechanisms.

30. The device of claim 28, wherein said maintaining said means to maintain said device in a selected position includes anchoring said device in a selected position.

31. The device of claim 28, wherein said delivering said guidewire includes steering said guidewire for delivery to at least one selected location.

32. The device of claim 28, wherein said means for delivering a guidewire to at least one pericardial location in a minimal invasive way comprises a means for locating a selected location in a pericardial space, using a needle having a sensing means to determine when the distal end of said needle is in said pericardial space; anchoring said needle in said pericardial space; and entering a guidewire to said selected location in said pericardial space, by guiding said guidewire through said needle.

33. The device of claim 32, comprising a plurality of guidewires for entering into a plurality of selected locations in a pericardial space, using said needle.

34. A device for delivering a guidewire to at least one pericardial location in a minimal invasive way, comprising: a needle having a sensing means to determine when the distal end of said needle is in said pericardial space; an anchoring means to secure said distal end of said needle in said pericardial space; and a guidewire to be delivered to said selected location in said pericardial space, said guidewire being deliverable through said needle.

35. A device for treating Bundle branch block in a minimal invasive way, comprising:
a means for locating at least one pericardial location using a delivery lumen; a means for anchoring said delivery lumen at said pericardial location; and a means for delivering at least one electrode to at least one pericardial location, using said locating means.

36. The device of claim 35, wherein said device for treating Bundle branch block in comprises a means for delivering at least one electrode to at least one pericardial location;
and generating one or more pulses using said at least one electrode, said pulses configured to synchronize cardiac electrical impulses.

37. The device of claim 36, wherein each of a plurality of said pulses is configured to optimally synchronize cardiac electrical impulses.
